(19) United States
(12) Patent Application Publication
(10) Pub. No.: US 2007/0000793 A1
(43) Pub. Date: Jan. 4, 2007

(54) Novelty dispenser package for adhesive tabs

(76) Inventor: Gunnard L. Nelson, Stilwell, KS (US)

Correspondence Address: HOVEY WILLIAMS LLP, 2405 GRAND BLVD., SUITE 400, KANSAS CITY, MO, 64108, US

(21) Appl. No.: 11/170,222

(22) Filed: Jun. 29, 2005

(51) Int. Cl. A61B 17/06

(52) U.S. Cl. 206/63.3

(57) ABSTRACT

Novelty dispensing packages (20, 66, 102) are provided which include a base (22, 68, 104) presenting one or more concavities (40, 86, 88) adapted to receive a stack (28, 74, 76, 110) of individually dispensable, image-bearing, adhesive tabs (30). The packages (20, 66, 102) also include preferably transparent upper covers (24, 70, 106) which mate with the bases (22, 68, 104), as well as image-bearing panels (26, 72, 108) which are sandwiched between the bases (22, 68, 104) and the covers (24, 70, 106). Preferably, the visible images on the panels (26, 72, 108) relate to the images provided on the individual tabs (30).

### BACKGROUND OF THE INVENTION

1. Field of the Invention

The present invention is concerned with novel dispenser packages allowing the ready, one-by-one dispensing of preprinted, adhesive-bearing tabs. More particularly the invention is concerned with such packages which can be easily designed and modified to convey desired images or advertising messages, without the need for complete redesign of the packages. The packages of the invention preferably include a preprinted, separate panel which mates with a transparent upper cover of the package, and the individual tabs are imprinted with images or text which is related to the panel design.

2. Description of the Prior Art

Elongated, adhesive tabs or flags have long been used as markers on typewritten sheets or the like. For example, tabs bearing the words “Sign Here” together with a pointer arrow are commonly applied to letters or other documents to make apparent where signatures are required. Generally, such tabs are formed paper stock or synthetic resin material and have a portion of the underside thereof covered with a removable or repositional adhesive. Such adhesive may be of the microsphere variety, or a pressure sensitive, hot-melt adhesive applied in the form of small spaced dots or islands.

In order to facilitate use of such tabs, they have commonly been provided in the form of stacks placed within specially designed dispenser packages. For example, U.S. Pat. No. 4,907,825 discloses a tab dispenser wherein the tab stack is situated within an elongated package permitting the stack to shuttle back and forth during dispensing of individual tabs. Normally, the top cover of such packages is substantially transparent, so that the user can see the tabs.

A problem with such prior tab dispensers is that only the tabs themselves are visible through the package cover. This inherently limits the nature and extent of the imagery and/or advertising message that can be conveyed with the package.

### SUMMARY OF THE INVENTION

The present invention overcomes the problems outlined above, and provides an improved dispenser package having a degree of design flexibility not achievable with prior packages. The packages of the invention include a base presenting an elongated concavity, together with a light-transmitting (preferably translucent or substantially transparent) cover disposed over the base and having a dispensing slot in communication with the base of concavity. A stack of elongated, adhesive-bearing tabs are located within the concavity beneath the cover and are constructed and oriented such that one end of the topmost tab extends through the dispensing slot, and ends of underlying tabs are pulled through the slot during dispensing of immediately proceeding tabs so that respective tabs of the stack can be successively dispensed through the slot. An imprinted panel is located beneath the cover and is visible therethrough, with the panel preferably being separate and structurally distinct from the cover and the tab stack, and of a size to substantially completely cover the area of the package cover.

In preferred form, the tabs are also imprinted with indicia related to that presented on the package panel. The tabs may be imprinted identically, or different tabs may have different indicia thereon. For example, the panel may be imprinted with an animal or human likeness whereas the tabs may be imprinted with indicia representing an anatomical feature of the animal or human likeness. Alternately, the package may be substantially rectangular and the size of a deck of cards; in such case the panel may be imprinted with playing card-related indicia whereas the tabs may be imprinted with different card suit and denomination indicia.

### BRIEF DESCRIPTION OF THE DRAWINGS

FIG. 1 is a front elevational view of a dispenser package in accordance with the invention, with the uppermost tab of a tab stack extending through the package dispensing slot in a first tab orientation;

FIG. 2 is a front elevational view similar to that of FIG. 1, but showing an alternate tab extending through the dispensing slot and assuming an orientation opposite to that of FIG. 1;

FIG. 3 is a sectional view taken along line 3-3 of FIG. 1;

FIG. 4 is a sectional view taken along line 4-4 of FIG. 2;

FIG. 5 is an exploded view of the components making up the dispenser package of FIGS. 1-4;

FIG. 6 is a perspective view of another dispenser package having a pair of side-by-side tab stacks, each with the uppermost tab extending through an associated package dispensing slot;

FIG. 7 is a fragmentary, enlarged vertical sectional view illustrating the construction of the FIG. 6 package and one of the tab stacks therein;

FIG. 8 is a fragmentary view illustrating the use of one of the tabs from the FIG. 6 dispenser, adhered to a sheet;

FIG. 8A is a plan view of a tab in accordance with the invention, showing the underside thereof having partial coverage with a repositionable adhesive;

FIG. 9 is an elevational view of another dispenser package in accordance with the invention; and

FIG. 10 is an enlarged, fragmentary sectional view taken along line 10-10 of FIG. 9.

### DETAILED DESCRIPTION OF THE PREFERRED EMBODIMENT

Turning now to the drawings, an exemplary dispenser package 20 is depicted in FIGS. 1-5. Broadly speaking, the package 20 includes a base 22, cover 24, imprinted panel 26 and tab stack 28. The package 20 is designed to permit successive dispensing of individual adhesives-bearing tabs 30 making up the stack 28. The tabs 30 can be releasably applied to sheets of paper or the like to give emphasis to a particular region of the sheet. This utility is depicted in FIG. 8 where a tab 30 is applied to the side margin of a sheet 31 as a marker or flag.

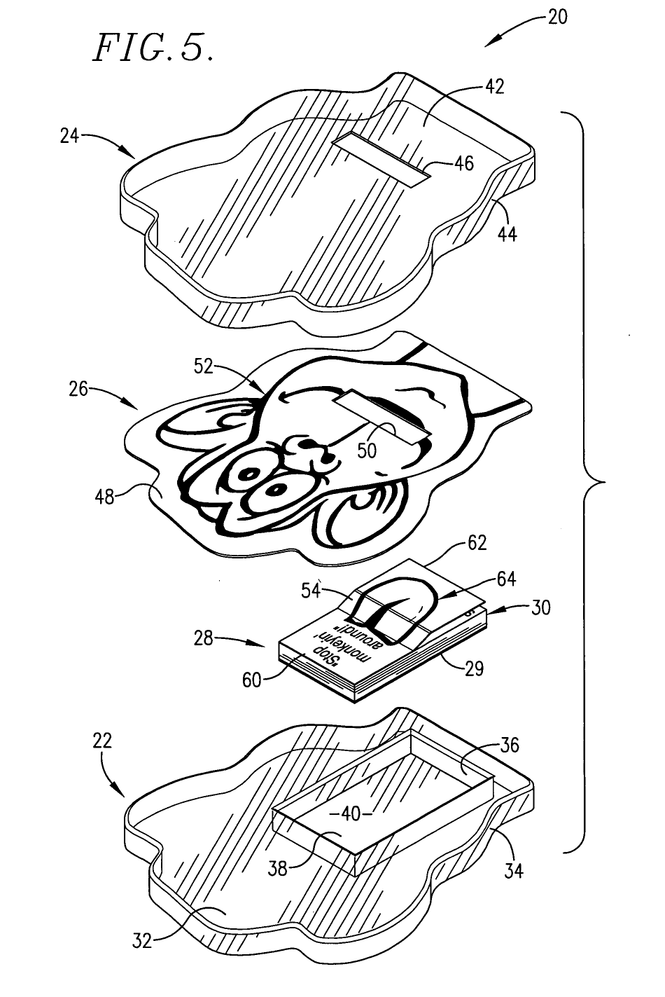

In more detail, the base 22 is integrally formed of substantially transparent vacuum formed or injection-molded synthetic resin material, and includes a top wall 32, depending peripheral sidewalls 34, interior, depending sidewalls 36 and a bottom wall 38 extending between the lower ends of the interior sidewalls 36. As best seen in FIG. 5, the interior sidewalls 36 cooperatively defined a substantially rectangular, open top concavity 40.

Cover 24 is designed to mate with base 22, and is likewise integrally formed of substantially transparent vacuum formed or injected-molded resin material. The cover 24 includes a top wall 42 and depending sidewalls 44; the latter are designed to snugly fit against sidewalls 34 of base 22 when the package 20 is assembled. The top wall 42 also includes a transversely oriented dispensing slot 46 which communicates with concavity 40 when the package is assembled.

The panel 26 is preferably formed of flexible material such paper stock or metallic material and has an imprinted upper surface 48. The peripheral contour of the panel conforms with the base 22 and cover 24, so that the panel essentially covers the underside of cover top wall 42. The panel has a dispensing slot 50 formed therein which is in registry with slot 46. In the depicted embodiment, the surface 48 is imprinted with a stylized monkey face 52, with the slot 50 located at the mouth region of the monkey face.

The stack 28 is made up of a plurality face-to-face adhesively interconnected tabs 30. Referring to FIG. 8 and 8a, each tab 30 is preferably of generally rectangular configuration and presents an upper imprinted face 54 and an opposed lower face 56. The lower face 56 has repositional or removable adhesive 58 applied to one end thereof so that the tab has an adhesive end 60 and a free end 62. As illustrated, the adhesive 58 covers only a minority of the surface area of lower face 56, typically on the order of 40-45% of the total surface area.

The tabs 30 are oriented so as to permit one-by-one dispensing thereof from stack 28. Thus, the tabs 30 are alternately arranged, i.e., the adhesive end 60 of each tab is adhered to the free end 62 of the next adjacent tab, on the imprinted upper face 54 thereof. The stack 28 is designed to be received within the base concavity 40, and is preferably adhered in place therein. During manufacture of the stack 28, material in roll form is preferably provided with strips of adhesive, using a gravure roller or the like, images are imprinted on the non-adhesive side of the roll material, the roll is cut into individual sheets, the sheets are collated, and the individual stacks are die cut from the collated sheets. In one embodiment of the invention the adhesive layer on the bottom most tab 30 of the stack 28 will secure the stack 28 to the bottom wall 38 of cavity 40. In the alternative, adhesive layer 29 of paper or plastic may be provided on the bottom tab of each stack 28, with a layer of adhesive being affixed to the outside surface of the stack 28 to adhesively affix stack 28 to bottom wall 38 of the cavity 40. It is preferred that the backing layer be incorporated with the sheets of the time of collation thereof. Thus, the backing layer 29 of each stack 28 is of the same dimensions as each sheet of the individual stacks 28. Additionally, the transverse end walls defining the cavity 40 are closely spaced relative to the ends of the stack 28. In this fashion, the stack 28 is held in place within the cavity 40 so as to prevent any substantial translatory movement of the stack.

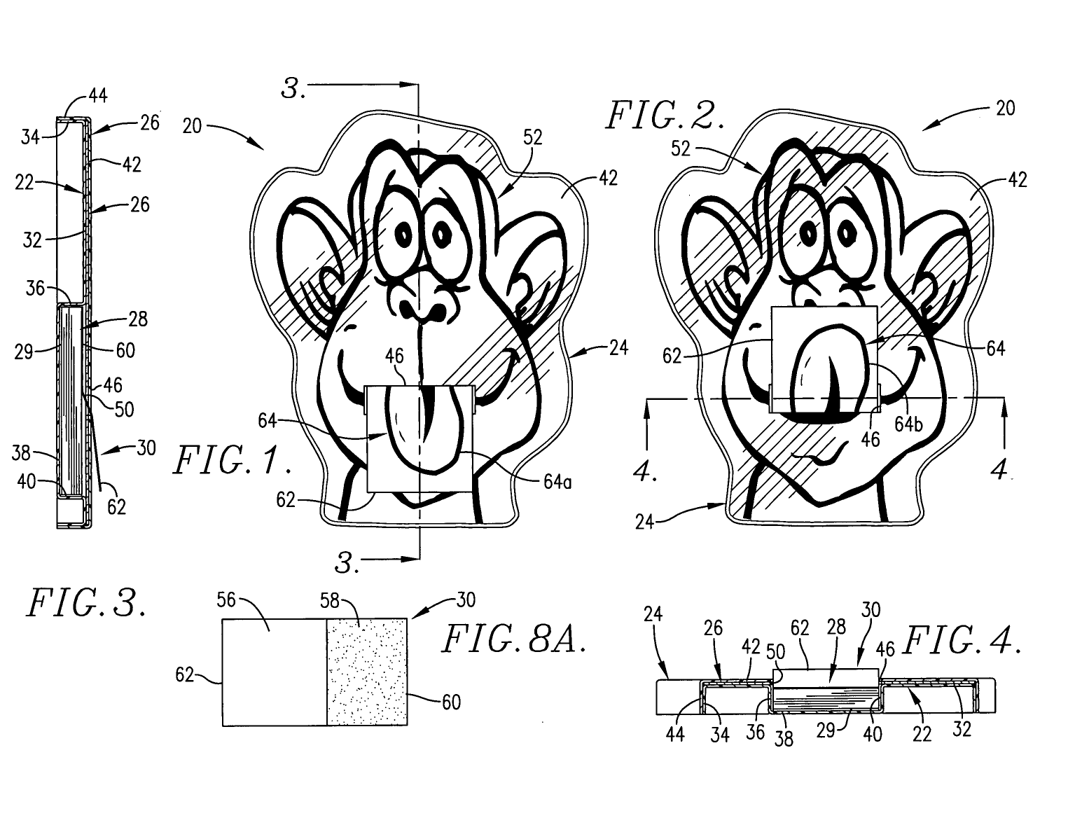

In the illustrated embodiment, the tabs 30 are each imprinted primarily on the free end 62 of upper face 54 with a stylized tongue 64 which relates to the monkey face 52 provided on panel 26. Preferably, the tabs 30 are alternately placed in stack 28 with a “tongue down” image 64a (FIG. 1) and a “tongue up” image 64b (FIG. 2). In addition, each of the tabs has an inscription on the upper face thereof at the adhesive end 60 which reads “Stop Monkeyin' Around.”

When the package is assembled as depicted in FIGS. 1-4, the panel 26 is sandwiched between top wall 38 of base 22 and the inner face of top wall 42 of cover 24. The slots 46 and 50 are in registry as shown. The stack 28 is seated and adhesively secured within concavity 40 with the free end 62 of the uppermost tab thereof extending through the slots 50, 46 for ready dispensing. In use, the free end 62 of the uppermost tab is grasped and pulled through the slots. This action serves to pull the free end 62 of the next adjacent tab 30 through the slots so as to make the next tab ready for dispensing. This causes the tabs to alternately present “tongue down” image 64a as shown in FIG. 1, and the “tongue up” image 64b as shown in FIG. 2.

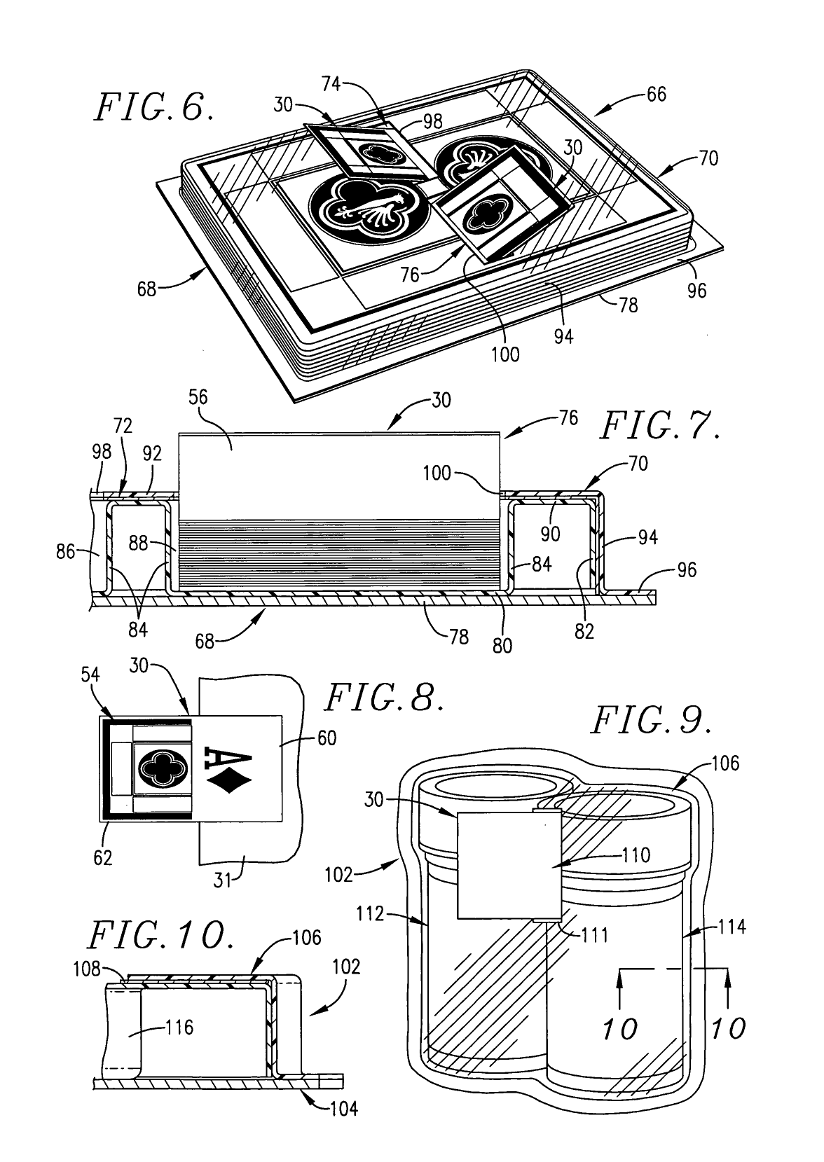

The invention is susceptible to a number of variations, depending upon the desired visual imagery and text. FIGS. 6-7 illustrate one such alternative in the form of a package 66 generally sized and configured to mimic a deck of cards. To this end, the package 66 includes a base 68, cover 70, imprinted panel 72 and two individual stacks 74 and 76 of imprinted tabs 30.

The base 68 has a lowermost panel 78 formed of cardboard paper stock or the like together with a vacuum formed or injection molded synthetic resin body 80 secured to the upper face of panel 78. The body 80 includes a peripheral sidewall 82, a top wall 90, and internal sidewalls 84 depending from top wall 90 that define a pair of spaced juxtaposed, open-top concavities 86 and 88.

The cover 70 includes a top wall 92 and a depending sidewall 94 terminating in a lower peripheral flange 96 secured to the upper face of panel 78. The cover 70 is integrally formed of substantially transparent resin material and has a pair of dispensing slots 98 and 100 formed therein, respectively in communication with the concavities 86 and 88.

The panel 72 is substantially rectangular and is designed to snugly fit within cover 70, in a sandwiched relationship between the later and top wall 90 of base body 80.

The stacks 74 and 76 are identical with the stack 28 previously described except that the upper faces 54 of the respective tabs are imprinted with different images. In particular, the tabs of the stacks 74, 76 have the upper faces 54 of the adhesive ends 60 (FIG. 8) thereof imprinted with different card suits and denomination indicia; correspondingly, the upper faces 54 of the free ends 62 are imprinted with generic playing card-related indicia. Thus, as the user draws individual tabs from the stacks 74 and 76, different card suit and denomination images are provided. In FIG. 8, the tab is imprinted with the Ace of Diamonds imagery. Other tabs within the stacks would bear different suit and denomination indicia.

A still further embodiment of the indicia is depicted in FIG. 9, with the invention having a different form of artwork or advertising image theron as a part of package 102. The package 102 as shown in FIG. 10 has a base 104 presenting a tab stack-receiving concavity 116 and includes a slotted, substantially transparent, vacuum formed or injection molded cover 106, and a slotted panel 108 imprinted with the selected image or images sandwiched between the underside of cover 106 and base 104. A stack 110 of imprinted tabs 30 are positioned within the base concavity 116 with the uppermost tab 30 extending outwardly through the aligned slots 111 of the panel 108 and cover 106. Further structural details of the package 102 are readily apparent from the drawings, and are similar to those of package 66.

In this embodiment, the package base, and panel are cooperatively designed and imprinted to represent a pair side-by-side medicament bottles 112 and 114, with the aligned slots 111 oriented adjacent the bottle tops and transverse to the longitudinal axes of the depicted bottles. The individual tabs 30 may be imprinted with a plain color or with indicia representing a type of medicament corresponding to that shown on the bottles 112, 114.

It will thus be seen that the invention affords a means of providing a variety of whimsical and/or advertising messages through proper selection of the configuration of the package components and the images imprinted on the panels and individual tabs. Those skilled in the art will appreciate that the invention is thus limited only by the imagination of the designer.

I claim:

1. A dispenser package comprising:
a base presenting an elongated concavity;
a light-transmitting cover disposed over said base and having a dispensing slot in communication with said concavity;
a stack of elongated tabs located within said concavity beneath said cover, said stack being constructed and oriented such that one end of the topmost tab thereof extends through said slot, and ends of underlying tabs are pulled through said slot during dispensing of preceding tabs, so that the respective tabs of said stack can be successively dispensed through said slot; and
an imprinted panel located beneath said cover and visible therethrough.

2. The package of claim 1, each of said tabs being imprinted.

3. The package of claim 2, at least certain of said tabs having imprinting different that the imprinting on other tabs of said stack.

4. The package of claim 2, there being a relationship between the imprinting on said panel and the imprinting on said tabs.

5. The package of claim 1, said base including a bottom wall, upstanding peripheral sidewalls, upstanding interior walls defining said concavity, and a top wall extending between the upper ends of said cavity-defining interior walls and the upper end of said peripheral sidewalls.

6. The package of claim 1, said slot being oriented transversely relative to the longitudinal axis of said concavity.

7. The package of claim 1, said panel having a slot formed therein in substantial registry with said cover slot.

8. The package of claim 1, each of said tabs having an underside with a repositionable adhesive applied to one end thereof so that the tab presents an adhesive-bearing end, and a free end.

9. The package of claim 8, said stack formed by placing said tabs so that the adhesive-bearing ends thereof are alternately at opposite ends of the stack and are adhered to the next underlying tab.

10. The package of claim 9, said stack being constructed and arranged so that the free ends of respective tabs extend through said slot, and such that when the topmost tab is dispensed by pulling it through said slot, the free end of the next underlying tab is pulled through the slot and is available for dispensing.

11. The package of claim 1, said panel being structurally distinct from said base and said cover.

12. The package of claim 1, said panel formed of flexible material.

13. The package of claim 1, said cover being substantially transparent.

14. The package of claim 1, said base having a top wall, said panel being sandwiched between said base top wall and said cover.

15. The package of claim 1, said concavity-defining walls including end walls configured for close adjacency with the ends of said stack in order to prevent any substantial translatory movement of the stack.

16. The package of claim 1, said base having a pair of concavities therein, there being an individual tab stack located within each concavity, said cover having a pair of dispensing slots each associated with one of said concavities.

17. The package of claim 1, said base and cover being substantially rectangular and of the size of a deck of cards, said panel being imprinted with card-related indicia, the tabs of said stack being imprinted with different card suit and denomination indicia.

18. The package of claim 1, said panel being imprinted with an animal or human likeness, the tabs of said stack being imprinted with indicia representing an anatomical feature of the animal or human likeness.
